(19) United States
(12) Patent Application Publication
(10) Pub. No.: US 2026/0294223 A1
(43) Pub. Date: Oct. 1, 2026

(54) ENDOSCOPE AND METHOD FOR MANUFACTURING ENDOSCOPE

(71) Applicant: KANEKA CORPORATION, Osaka (JP)

(72) Inventor: Shoma Sonohara, Nagano (JP)

(73) Assignee: KANEKA CORPORATION, Osaka (JP)

(21) Appl. No.: 19/252,841

(22) Filed: Jun. 27, 2025

Related U.S. Application Data
(63) Continuation of application No. PCT/JP2023/042532, filed on Nov. 28, 2023.

(30) Foreign Application Priority Data
Jan. 5, 2023 (JP) 2023-000667

(51) Int. Cl. A61B 1/05; A61B 1/00; A61B 1/06; A61B 1/07

(52) U.S. Cl. CPC A61B 1/05; A61B 1/0011; A61B 1/00119; A61B 1/0669; A61B 1/07

(57) ABSTRACT

An endoscope capable of brightly illuminating a body cavity, and a method for manufacturing the endoscope are disclosed. The endoscope has an imaging element, a light-emitting part, and a transparent resin member that is fixed to the imaging element and the light-emitting part and through which light emitted from the light-emitting part is capable of passing. The transparent resin member has, in a region having a length of 0.1 mm in a longitudinal axis direction of the endoscope, one or more and ten or fewer bubbles each having a major axis of 50 μm or more and 500 μm or less.

### TECHNICAL FIELD

One or more embodiments of the present invention relate to an endoscope having an imaging element, a light-emitting part, and a transparent resin member, and to a method for manufacturing an endoscope.

### BACKGROUND

Conventionally, there has been an endoscope that is provided, at its distal end, with an objective lens and an illumination lens, and that is inserted into a human body to observe a body cavity while allowing a treatment tool to be introduced into the body cavity via a treatment tool insertion channel extending from a proximal side to the distal side. Medical procedures performed using such an endoscope include, for example, ESD (Endoscopic Submucosal Dissection) and EMR (Endoscopic Mucosal Resection).

For example, Patent document 1 discloses an endoscopic device having a substrate disposed at a distal end portion of an elongated insertion section; an illumination light source mounted on the substrate; a wiring pattern that is electrically connected to the illumination light source and provided on the substrate; a covering part that is provided on the substrate so as to cover the wiring pattern, the covering part being light-transmissive and having insulating properties; and a sealing resin that is laminated on the covering part and is light-transmissive. Patent document 2 discloses an electronic endoscope having a hub; a shaft extending from the hub; an expandable distal tip portion extending from the shaft; an image sensor located inside the distal tip portion and having an external field of view from the endoscope; an illumination element located inside the distal tip portion and configured to emit light within the field of view of the image sensor; and at least one shape-variable working channel located inside the distal tip portion, the working channel being adapted to change its overall cross-sectional shape from a non-circular shape to a different shape to accommodate the passage of instruments. Patent document 3 discloses an endoscope having an endoscope head; a wide-angle lens located at a distal end of the endoscope head; and a working channel extending within the endoscope head, wherein an illumination means is disposed at the distal end of the endoscope head adjacent to the wide-angle lens. Patent document 4 discloses an endoscope distal end portion having a camera; a housing element made of a light-transmissive material that at least partially surrounds the camera laterally and extends to the distal end of the endoscope distal end portion; and at least one illumination part disposed in proximity to the camera, wherein the at least one illumination part may be located directly behind the camera when viewed from the distal side, and the housing element is made of a transparent plastic material or glass.

### PATENT DOCUMENTS

Patent document 1: JP 2013-230186 A

Patent document 2: JP 2018-525197 T

Patent document 3: JP 2020-503957 T

Patent document 4: JP 2022-505913 T

In the endoscopes disclosed in Patent documents 1 to 4, the brightness of the illumination provided by the endoscope may be insufficient, making it difficult to observe inside a body cavity such as the gastrointestinal tract.

One or more embodiments of the present invention have been made in view of the above circumstances, and to provide an endoscope and a method for manufacturing an endoscope, which are capable of brightly illuminating a body cavity and facilitating observation inside the body cavity.

### SUMMARY

An endoscope according to one or more embodiments of the present invention is as follows.
[1] An endoscope comprising:
an imaging element;
a light-emitting part; and
a transparent resin member that is fixed to the imaging element and the light-emitting part and through which light emitted from the light-emitting part is capable of passing, wherein
the transparent resin member has, in a region having a length of 0.1 mm in a longitudinal axis direction of the endoscope, one or more and ten or fewer bubbles each having a major axis of 50 μm or more and 500 μm or less.
[2] The endoscope according to [1], wherein, in a cross section perpendicular to the longitudinal axis direction at a midpoint of a length of the transparent resin member in the longitudinal axis direction of the endoscope, the transparent resin member includes a central region defined by a circle centered at a centroid of an outer shape of the transparent resin member and having a diameter equal to half of a minor axis length of the outer shape of the transparent resin member, and a peripheral region excluding the central region; and
the number of the bubbles present in the central region is greater than the number of the bubbles present in the peripheral region.
[3] The endoscope according to [1] or [2], wherein the transparent resin member is located on a proximal side relative to a distal end of the imaging element and on a distal side relative to a proximal end of the light-emitting part;
the transparent resin member includes a distal region positioned on a side of the imaging element, a proximal region positioned on a side of the light-emitting part, and an intermediate region positioned proximal to the distal region and distal to the proximal region;
the number of the bubbles present in the intermediate region is greater than the number of the bubbles present in the distal region; and
the number of the bubbles present in the proximal region is greater than the number of the bubbles present in the intermediate region.
[4] The endoscope according to any one of [1] to [3], wherein a material constituting the transparent resin member includes an ultraviolet-curable resin.
[5] The endoscope according to any one of [1] to [4], further comprising a tubular body having an inner lumen and capable of accommodating the imaging element, the transparent resin member, and the light-emitting part in the inner lumen, wherein
in a state in which the imaging element, the transparent resin member, and the light-emitting part are exposed from the tubular body, the transparent resin member has a contact surface on its outer surface, the contact surface being capable of contacting a wall of a biological lumen.
[6] The endoscope according to any one of [1] to [5], wherein a reflective material that reflects light emitted from the light-emitting part is disposed inside the transparent resin member, the reflective material being located on a proximal side relative to the imaging element and on a distal side relative to the light-emitting part.
[7] The endoscope according to any one of [1] to [6], wherein an amount of ultraviolet light included in light emitted from the light-emitting part is 1 μW/cm² or less.
[8] The endoscope according to any one of [1] to [7], further comprising a covering tube located on a proximal side relative to a distal end of the light-emitting part, wherein
a core member is disposed inside the transparent resin member, and
the core member extends from at least a proximal end of the imaging element to a position proximal to a distal end of the covering tube.
[9] The endoscope according to any one of [1] to [8], wherein at least one of the imaging element and the light-emitting part has a groove that extends in a direction perpendicular to a longitudinal axis direction of the endoscope.
[10] The endoscope according to any one of [1] to [9], wherein, in a cross section perpendicular to a longitudinal axis direction of the endoscope, the imaging element and the light-emitting part each have a rectangular cross-sectional shape, and the transparent resin member has a circular cross-sectional shape.
[11] The endoscope according to any one of [1] to [10], wherein an average value of a surface roughness Ra of the transparent resin member is 25 μm or less.

One or more embodiments of the present invention also provide a method for manufacturing an endoscope. A manufacturing method according to one or more embodiments of the present invention is as follows.
[12] A method for manufacturing an endoscope having an imaging element, a light-emitting part, and a transparent resin member through which light emitted from the light-emitting part is capable of passing and which has, in a region having a length of 0.1 mm in a longitudinal axis direction, one or more and ten or fewer bubbles each having a major axis of 50 μm or more and 500 μm or less, the method comprising:
placing the imaging element and the light-emitting part in an inner lumen of a heat-shrinkable tube;
heating the heat-shrinkable tube;
filling the inner lumen of the heat-shrinkable tube with a transparent liquid resin that is curable by ultraviolet irradiation;
forming bubbles in the transparent liquid resin;
irradiating the transparent liquid resin with ultraviolet light to form the transparent resin member; and
removing the heat-shrinkable tube.
[13] The manufacturing method according to [12], wherein
the heat-shrinkable tube is capable of transmitting ultraviolet light, and
in the step of irradiating the transparent liquid resin with ultraviolet light to form the transparent resin member, the ultraviolet light is irradiated from outside the heat-shrinkable tube.
[14] The manufacturing method according to [12] or [13], wherein,
in the step of filling the inner lumen of the heat-shrinkable tube with the transparent liquid resin, a core member is disposed in the inner lumen of the heat-shrinkable tube, and
in the step of forming bubbles in the transparent liquid resin, the core member is moved.
[15] The manufacturing method according to any one of [12] to [14], further comprising:
before filling the inner lumen of the heat-shrinkable tube with the transparent liquid resin, placing a covering tube on a proximal side relative to the light-emitting part and inserting a wire of the imaging element and a wire of the light-emitting part into an inner lumen of the covering tube, and
before irradiating the transparent liquid resin with ultraviolet light to form the transparent resin member, filling the transparent liquid resin into the inner lumen at a distal end of the covering tube.

According to the endoscope of one or more embodiments of the present invention, since the transparent resin member has, in a region having a length of 0.1 mm in a longitudinal axis direction of the endoscope, one or more and ten or fewer bubbles each having a major axis of 50 μm or more and 500 μm or less, light emitted from the light-emitting part is diffused by the bubbles in the transparent resin member as it passes through the transparent resin member. Therefore, a wide area inside a body cavity can be brightly illuminated, making it easier to observe the interior of the body cavity using the endoscope.

### BRIEF DESCRIPTION OF THE DRAWINGS

FIG. 1 is an enlarged plan view of a distal portion of an endoscope according to one or more embodiments of the present invention.

FIG. 2 is a sectional view taken along line II-II of the endoscope shown in FIG. 1.

FIG. 3 is a sectional view taken along line III-III of the endoscope shown in FIG. 1.

FIG. 4 is a sectional view taken along line IV-IV of the endoscope shown in FIG. 1.

FIG. 5 is a schematic view (partially in sectional view) illustrating a step of placing an imaging element and a light-emitting part in an inner lumen of a heat-shrinkable tube according to one or more embodiments of the present invention.

FIG. 6 is a schematic view (partially in sectional view) illustrating a step of heating the heat-shrinkable tube according to one or more embodiments of the present invention.

FIG. 7 is a schematic view (partially in sectional view) illustrating a step of filling a transparent liquid resin into an inner lumen of the heat-shrinkable tube according to one or more embodiments of the present invention.

FIG. 8 is a schematic view (partially in sectional view) illustrating a step of forming bubbles in the transparent liquid resin according to one or more embodiments of the present invention.

FIG. 9 is a schematic view illustrating a step of removing the heat-shrinkable tube according to one or more embodiments of the present invention.

FIG. 10 is a schematic view (partially in sectional view) illustrating a step of placing a wire of the imaging element and a wire of the light-emitting part in an inner lumen of a covering tube according to one or more embodiments of the present invention.

### DETAILED DESCRIPTION

Hereinafter, the present disclosure will be described based on the following embodiments, however, the present disclosure is not limited by the following embodiments and can be altered in design within a scope in compliance with the intent described above and below, and all the changes are to be encompassed within a technical scope of the present disclosure. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of one or more embodiments of the present invention, the dimensions may differ from the actual dimensions in some cases.

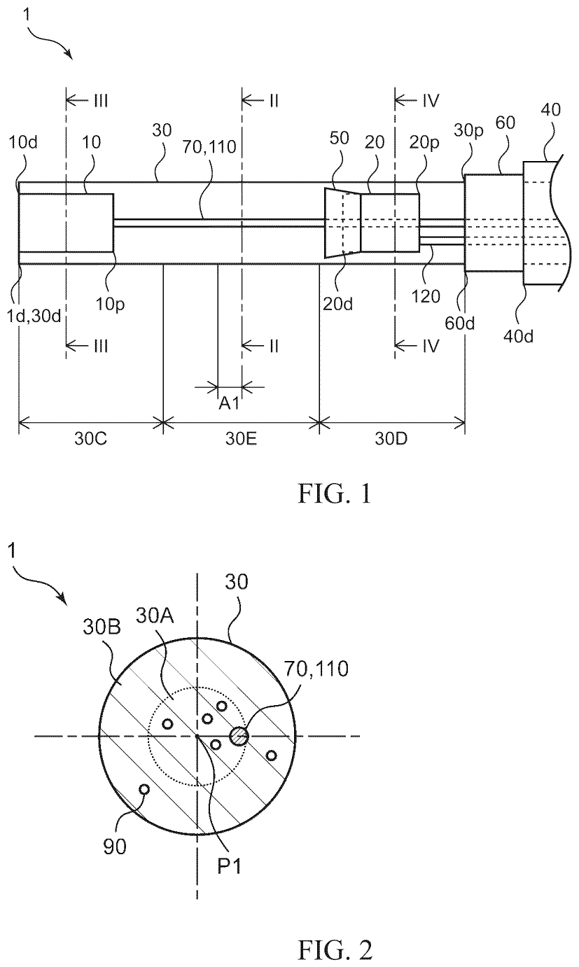

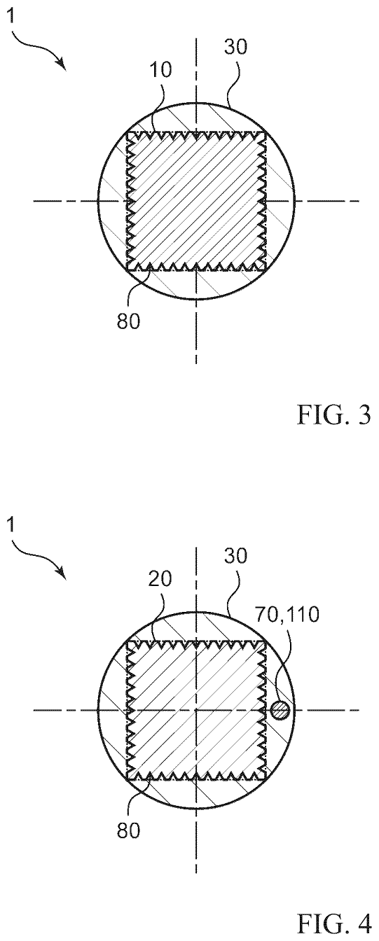

First, an endoscope of one or more embodiments of the present invention will be described. FIG. 1 is an enlarged plan view of a distal portion of an endoscope 1 according to one or more embodiments of the present invention, and FIGS. 2, 3, and 4 are sectional views of the endoscope 1. Specifically, FIG. 2 is a cross-sectional view taken in a plane perpendicular to the longitudinal axis direction at a midpoint of a length of a transparent resin member 30 of the endoscope 1, FIG. 3 is a cross-sectional view of an imaging element 10 taken in a plane perpendicular to the longitudinal axis direction of the endoscope 1, and FIG. 4 is a cross-sectional view of a light-emitting part 20 taken in a plane perpendicular to the longitudinal axis direction of the endoscope 1.

In the present disclosure, the proximal side refers to the side closer to the user's hand along the extending direction of the endoscope 1, and the distal side refers to the side opposite to the proximal side, that is, the side toward the treatment target. The extending direction of the endoscope 1 is referred to as the longitudinal axis direction. In FIG. 1, the right side of the drawing corresponds to the proximal side, and the left side corresponds to the distal side.

As shown in FIG. 1, an endoscope 1 of one or more embodiments of the present invention includes an imaging element 10 and a light-emitting part 20, and has a transparent resin member 30 that is fixed to the imaging element 10 and the light-emitting part 20 and through which light emitted from the light-emitting part 20 is capable of passing.

Preferably, the endoscope 1 is capable of observing biological lumens such as the gastrointestinal tract including the esophagus, stomach, small intestine, and large intestine; blood vessels such as coronary arteries; respiratory organs such as the thoracic cavity and bronchi; urinary organs such as the bladder and renal pelvis; the pancreas; and biological lumens such as the bile duct.

Preferably, the endoscope 1 is a disposable endoscope. By making the endoscope 1 disposable, it is possible to prevent infection of the subject being observed through the endoscope 1 and to improve safety.

The imaging element 10 converts light from a subject into an electrical signal or the like and outputs it. Examples of the imaging element 10 include a CCD (Charge Coupled Device) and a CMOS (Complementary Metal-Oxide Semiconductor). By transmitting an electrical signal from the imaging element 10 via a wire 110 of the imaging element 10 to an image display device such as an external video processor, it is possible to confirm an image of the inside of the body on the image display device.

Preferably, a lens is provided on the imaging element 10. When a lens is provided on the imaging element 10, the lens of the imaging element 10 may be positioned at a distal end 1d of the endoscope 1. By providing a lens on the imaging element 10, it becomes easier to collect light from a subject, thereby facilitating observation of a biological lumen using the endoscope 1.

The light-emitting part 20 emits light. Examples of the light-emitting part 20 include light sources such as a light-emitting diode (LED), a xenon lamp, and an organic light-emitting diode (organic EL), or components such as a core of an optical fiber.

Although not shown in the figures, when a light source such as an LED, a xenon lamp, or an organic EL is used as the light-emitting part 20, the light-emitting part 20 may be connected to a power supply device that supplies electricity to the light-emitting part 20 via a wire 120 of the light-emitting part 20. By connecting the light-emitting part 20 to the power supply device via the wire 120, electricity is supplied from the power supply device through the wire 120 to the light source that constitutes the light-emitting part 20, thereby enabling the light-emitting part 20 to emit light.

Although not shown in the figures, when an optical fiber is used as the light-emitting part 20, one end of the optical fiber may be connected to a light source device such as an LED, a xenon lamp, or an organic EL. When one end of the optical fiber is connected to the light source device, light emitted from the light source device enters the optical fiber from the one end, and the incident light travels through the core of the optical fiber and is emitted from the other end of the optical fiber. As a result, the light-emitting part 20 is capable of emitting light.

Although not shown in the figures, when an optical fiber is used as the light-emitting part 20, one end of the optical fiber that is connected to the light source device may be located on a proximal side relative to the other end of the optical fiber. When one end of the optical fiber is located on a proximal side relative to the other end, light from the light source device that enters the optical fiber from the one end travels toward the distal side, and it becomes easier to observe a region distal to the position where the distal end 1d of the endoscope 1 is located, as the light illuminates that distal region.

Light emitted from the light-emitting part 20 may be visible light. That is, the wavelength of the light emitted from the light-emitting part 20 may be 360 nm or more and 830 nm or less. This means that it is preferable for the light emitted from the light-emitting part 20 to include light having a wavelength of 360 nm or more and 830 nm or less. For example, the light-emitting part 20 may temporarily emit light having a wavelength of less than 360 nm, or may temporarily emit light having a wavelength exceeding 830 nm. Among these, more preferably, the light emitted from the light-emitting part 20 consists only of light having a wavelength of 360 nm or more and 830 nm or less. When the light emitted from the light-emitting part 20 consists only of light having a wavelength of 360 nm or more and 830 nm or less, it becomes possible to make the endoscope 1 suitable for easier observation of a body cavity.

The transparent resin member 30 allows light emitted from the light-emitting part 20 to pass through. In the present disclosure, “transparent” means that light is transmitted through. The transparent resin member 30 may be either transparent or translucent, as long as light is able to pass through it.

The transparent resin member 30 may be either uncolored or colored, as long as it has light-transmitting properties. Among these, the transparent resin member 30 may be uncolored. When the transparent resin member 30 is uncolored, light emitted from the light-emitting part 20 and passing through the transparent resin member 30 does not become tinted, making it easier to observe the interior of a body cavity.

The transparent resin member 30 may be capable of transmitting 70% or more of visible light emitted from the light-emitting part 20, 75% or more, or 80% or more. By setting the lower limit of the percentage of visible light that the transparent resin member 30 is capable of transmitting within the above range, the transparent resin member 30 is less likely to hinder the passage of light emitted from the light-emitting part 20, making it easier to brightly illuminate the interior of a body cavity. The upper limit of the percentage of visible light that the transparent resin member 30 is capable of transmitting is not particularly limited and may be, for example, 100% or less.

As shown in FIG. 1 and FIG. 2, the transparent resin member 30 has, in a region A1 having a length of 0.1 mm in the longitudinal axis direction of the endoscope 1, one or more and ten or fewer bubbles 90 each having a major axis of 50 μm or more and 500 μm or less. In other words, the transparent resin member 30 contains, in the region A1 having a length of 0.1 mm in the longitudinal axis direction of the endoscope 1, one or more and ten or fewer bubbles 90 each having a major axis of 50 μm or more and 500 μm or less. Stated differently, the transparent resin member 30 has the region A1 in which, in a length of 0.1 mm in the longitudinal axis direction of the endoscope 1, one or more and ten or fewer bubbles 90 each having a major axis of 50 μm or more and 500 μm or less are included.

When the transparent resin member 30 has, in the region A1 having a length of 0.1 mm in the longitudinal axis direction of the endoscope 1, one or more and ten or fewer bubbles 90 each having a major axis of 50 μm or more and 500 μm or less, light emitted from the light-emitting part 20 is diffusely reflected and scattered by the bubbles 90 present in the transparent resin member 30 as it passes through the transparent resin member 30. As a result, the light emitted from the light-emitting part 20 is able to brightly illuminate a wide area inside the body cavity, making it easier to observe the interior of the body cavity with the endoscope 1.

The method for measuring the number of the bubbles 90 present in the region A1 of the transparent resin member 30 is as follows. At an arbitrary location of the transparent resin member 30, the transparent resin member 30 is cut so that its thickness becomes 0.1 mm, and a measurement sample is prepared. The cross section of the measurement sample is observed using a microscope or the like, and the number of the bubbles 90 having a major axis of 50 μm or more and 500 μm or less in the cross section of the measurement sample is counted. In the present disclosure, the term “bubble 90” refers to a bubble whose major axis is 50 μm or more and 500 μm or less in the cross section of the measurement sample. Stated differently, in the cross section of the measurement sample, bubbles whose major axis is less than 50 μm or greater than 500 μm are not regarded as the bubbles 90 of the present disclosure.

The number of the bubbles 90 having a major axis of 50 μm or more and 500 μm or less that the transparent resin member 30 has in the region A1 having a length of 0.1 mm in the longitudinal axis direction of the endoscope 1 may be two or more, three or more, or four or more. By setting the lower limit of the number of the bubbles 90 present in the region A1 of the transparent resin member 30 within the above range, the light emitted from the light-emitting part 20 is more easily scattered by the bubbles 90 of the transparent resin member 30, making it easier to brightly illuminate the interior of a body cavity. As a result, the interior of the body cavity can be more easily observed with the endoscope 1. In addition, the number of the bubbles 90 having a major axis of 50 μm or more and 500 μm or less that the transparent resin member 30 has in the region A1 may be nine or fewer, eight or fewer, or seven or fewer. By setting the upper limit of the number of the bubbles 90 present in the region A1 within the above range, attenuation of the light emitted from the light-emitting part 20 due to the bubbles 90 in the transparent resin member 30 is less likely to occur, and the amount of light emitted from the light-emitting part 20 is less likely to decrease.

In the transparent resin member 30, the region A1 in which one or more and ten or fewer of the bubbles 90 having a major axis of 50 μm or more and 500 μm or less are present may be a location where the imaging element 10 and the light-emitting part 20 are not disposed. When the region A1 is a location where the imaging element 10 and the light-emitting part 20 are not disposed, the number of the bubbles 90 can be measured more easily, and the light emitted from the light-emitting part 20 and passing through the transparent resin member 30 is more easily scattered by the bubbles 90.

As shown in FIG. 1, the imaging element 10 may be disposed at the distal end 1d of the endoscope 1. The imaging element 10 disposed at the distal end 1d of the endoscope 1 facilitates observation of a region distal to the distal end 1d of the endoscope 1.

As shown in FIG. 1, the light-emitting part 20 may be disposed on a proximal side relative to the imaging element 10. The light-emitting part 20 disposed on the proximal side relative to the imaging element 10 can irradiate light toward a distal side from the proximal side of the imaging element 10. Consequently, the imaging element 10 readily captures light from a subject, facilitating observation inside a body cavity with the endoscope 1.

The distal end 30d of the transparent resin member 30 may be located on a distal side relative to the proximal end 10p of the imaging element 10. In other words, the transparent resin member 30 may be present outside the imaging element 10. When the distal end 30d of the transparent resin member 30 is located on the distal side of the proximal end 10p of the imaging element 10, the transparent resin member 30 is present around the imaging element 10 at the proximal end 10p of the imaging element 10. Therefore, it is possible to increase the contact area between an outer surface of the imaging element 10 and the transparent resin member 30, thereby facilitating enhancement of bonding strength and making the imaging element 10 less likely to come off the endoscope 1.

The distal end 30d of the transparent resin member 30 may be present at least up to the distal end 10d of the imaging element 10. When the distal end 30d of the transparent resin member 30 is present at least up to the distal end 10d of the imaging element 10, the entire outer surface of the imaging element 10 readily comes into contact with the transparent resin member 30. As a result, the area of contact between the imaging element 10 and the transparent resin member 30 increases, and the bonding strength between the imaging element 10 and the transparent resin member 30 tends to increase.

The proximal end 30p of the transparent resin member 30 may be located on a proximal side relative to the distal end 20d of the light-emitting part 20. In other words, the transparent resin member 30 may be present outside the light-emitting part 20. When the proximal end 30p of the transparent resin member 30 is located on the proximal side of the distal end 20d of the light-emitting part 20, the transparent resin member 30 is present around the light-emitting part 20 at the distal end 20d of the light-emitting part 20. As a result, the area of contact between an outer surface of the light-emitting part 20 and the transparent resin member 30 can be increased, thereby facilitating enhancement of the bonding strength between the light-emitting part 20 and the transparent resin member 30 and making the light-emitting part 20 less likely to come off.

The proximal end 30p of the transparent resin member 30 may be present at least up to the proximal end 20p of the light-emitting part 20. In other words, the proximal end 30p of the transparent resin member 30 may be located at the proximal end 20p of the light-emitting part 20, and may also be located on a proximal side relative to the proximal end 20p of the light-emitting part 20. When the proximal end 30p of the transparent resin member 30 is present at least up to the proximal end 20p of the light-emitting part 20, the entire outer surface of the light-emitting part 20 is more likely to come into contact with the transparent resin member 30, thereby making it easier to enhance the bonding strength between the light-emitting part 20 and the transparent resin member 30.

As shown in FIG. 2, in a cross section perpendicular to the longitudinal axis direction at the midpoint of the length of the transparent resin member 30 in the longitudinal axis direction of the endoscope 1, the transparent resin member 30 includes a central region 30A defined by a circle centered at a centroid P1 of the outer shape of the transparent resin member 30 and having a diameter equal to half of a minor axis length of the outer shape of the transparent resin member 30, and a peripheral region 30B excluding the central region 30A. The number of the bubbles 90 present in the central region 30A may be greater than the number of the bubbles 90 present in the peripheral region 30B.

The central region 30A is a region inside a circle that is defined, in the cross section perpendicular to the longitudinal axis direction at the midpoint of the length of the transparent resin member 30 in the longitudinal axis direction of the endoscope 1, by being centered at the centroid P1 of the outer shape of the transparent resin member 30 and having a diameter equal to half of the minor axis length of the outer shape of the transparent resin member 30. The peripheral region 30B is a region excluding the central region 30A from the inner area of the outer shape of the transparent resin member 30 in the cross section perpendicular to the longitudinal axis direction at the midpoint of the length of the transparent resin member 30.

In the cross section perpendicular to the longitudinal axis direction at the midpoint of the length of the transparent resin member 30 in the longitudinal axis direction of the endoscope 1, when the number of the bubbles 90 present in the central region 30A is greater than the number of the bubbles 90 present in the peripheral region 30B, the strength of the peripheral region 30B of the transparent resin member 30 can be made higher than that of the central region 30A. As a result, in the transparent resin member 30 having the bubbles 90, even when a distal end portion of the endoscope 1 is in a bent state or the like, since the surface or the vicinity of the surface of the transparent resin member 30 tends not to contain the bubbles 90, the transparent resin member 30 around the bubbles 90 is less likely to be damaged, thereby preventing cracks from forming in the peripheral region 30B or fragments from coming off due to such damage. Furthermore, the light emitted from the light-emitting part 20 is more likely to be diffusely reflected by the bubbles 90 present in the central region 30A, thereby diffusing the light to brightly illuminate the interior of a body cavity.

In the cross section perpendicular to the longitudinal axis direction at the midpoint of the length of the transparent resin member 30 in the longitudinal axis direction of the endoscope 1, the central region 30A may include the bubbles 90, and the peripheral region 30B may be configured not to include the bubbles 90. When the central region 30A includes the bubbles 90 and the peripheral region 30B does not include the bubbles 90, it is possible for the central region 30A to produce the effect of diffusing the light emitted from the light-emitting part 20 to brightly illuminate the interior of a body cavity, and at the same time, the strength of the peripheral region 30B can be further increased, making the transparent resin member 30 less likely to be damaged.

In the cross section perpendicular to the longitudinal axis direction at the midpoint of the length of the transparent resin member 30 in the longitudinal axis direction of the endoscope 1, the number of the bubbles 90 present in the central region 30A may be 1.2 times or more the number of the bubbles 90 present in the peripheral region 30B, 1.5 times or more, or 2.0 times or more. By setting the lower limit of the ratio of the number of the bubbles 90 in the central region 30A to the number of the bubbles 90 in the peripheral region 30B within the above range, it is possible to increase the number of the bubbles 90 in the central region 30A relative to the peripheral region 30B, thereby making it easier for the central region 30A to diffuse the light emitted from the light-emitting part 20. Although the upper limit of the ratio of the number of the bubbles 90 in the central region 30A to the number of the bubbles 90 in the peripheral region 30B is not particularly limited, it may be, for example, 30 times or less, 20 times or less, or 10 times or less.

As shown in FIG. 1, the transparent resin member 30 may be located on a proximal side relative to the distal end 10d of the imaging element 10 and on a distal side relative to the proximal end 20p of the light-emitting part 20. In other words, at least a portion of the transparent resin member 30 may be present between the imaging element 10 and the light-emitting part 20 in the longitudinal axis direction of the endoscope 1. When the transparent resin member 30 is located on the proximal side of the distal end 10d of the imaging element 10 and on the distal side of the proximal end 20p of the light-emitting part 20, the proximal portion of the imaging element 10 and the distal portion of the transparent resin member 30 are fixed to each other, and the proximal portion of the transparent resin member 30 and the distal portion of the light-emitting part 20 are also fixed to each other. Accordingly, the imaging element 10, the transparent resin member 30, and the light-emitting part 20 are more likely to be firmly fixed together, making it less likely for the imaging element 10 or the light-emitting part 20 to come off from the transparent resin member 30. In addition, when the transparent resin member 30 is located on the proximal side of the distal end 10d of the imaging element 10 and on the distal side of the proximal end 20p of the light-emitting part 20, the light-emitting part 20 can emit light from the proximal side toward the distal side of the imaging element 10. As a result, the imaging element 10 can more easily receive light from a subject, thereby making it easier to observe the interior of a body cavity using the endoscope 1.

Preferably, the transparent resin member 30 includes a distal region 30C positioned on the side of the imaging element 10, a proximal region 30D positioned on the side of the light-emitting part 20, and an intermediate region 30E positioned proximal to the distal region 30C and distal to the proximal region 30D; and the number of the bubbles 90 present in the intermediate region 30E is greater than the number of the bubbles 90 present in the distal region 30C, and the number of the bubbles 90 present in the proximal region 30D is greater than the number of the bubbles 90 present in the intermediate region 30E. In other words, among the distal region 30C, the intermediate region 30E, and the proximal region 30D in the transparent resin member 30 having the bubbles 90, the proximal region 30D may include the greatest number of the bubbles 90.

Preferably, the distal region 30C is a region located on the imaging element 10 side, that is, on the distal side, when the transparent resin member 30 is divided into three equal parts in the longitudinal axis direction of the endoscope 1; the proximal region 30D is a region located on the light-emitting part 20 side, that is, on the proximal side, when the transparent resin member 30 is divided into three equal parts in the longitudinal axis direction of the endoscope 1; and the intermediate region 30E is a region located between the distal region 30C and the proximal region 30D, that is, in the middle, when the transparent resin member 30 is divided into three equal parts in the longitudinal axis direction of the endoscope 1.

By configuring the transparent resin member 30 such that the number of the bubbles 90 present in the intermediate region 30E is greater than the number of the bubbles 90 present in the distal region 30C, and the number of the bubbles 90 present in the proximal region 30D is greater than the number of the bubbles 90 present in the intermediate region 30E, the proximal region 30D includes the greatest number of the bubbles 90, followed by the intermediate region 30E. In the transparent resin member 30, by having a configuration in which the proximal region 30D includes the greatest number of the bubbles 90 and the intermediate region 30E includes the next greatest number of the bubbles 90, the number of the bubbles 90 increases as the location approaches the light-emitting part 20 that emits light, making the light emitted from the light-emitting part 20 more likely to be diffusely reflected by the bubbles 90 and thereby more easily diffused. As a result, the interior of a body cavity can be brightly illuminated, making it easier to observe the interior of the body cavity using the endoscope 1.

Preferably, the transparent resin member 30 has a configuration in which the number of the bubbles 90 present in the proximal region 30D is the greatest, and the number of the bubbles 90 present in the distal region 30C is the smallest. By configuring the transparent resin member 30 such that the number of the bubbles 90 present in the proximal region 30D is greater than the number of the bubbles 90 present in both the distal region 30C and the intermediate region 30E, the number of the bubbles 90 in the transparent resin member 30 near the light-emitting part 20 becomes greater than the number of the bubbles 90 in the transparent resin member 30 near the imaging element 10. As a result, it becomes possible to provide the endoscope 1 with a structure in which the light emitted from the light-emitting part 20 can be more easily diffused.

Preferably, a material constituting the transparent resin member 30 includes an ultraviolet-curable resin. By including an ultraviolet-curable resin in the material constituting the transparent resin member 30, it becomes possible to allow light to pass through while increasing the strength of the transparent resin member 30.

A material constituting the transparent resin member 30 may contain 85% or more of an ultraviolet-curable resin, 90% or more, or 95% or more. By setting the lower limit of the content of the ultraviolet-curable resin in the material constituting the transparent resin member 30 within the above range, it becomes easier to increase the strength of the distal end portion of the endoscope 1. The upper limit of the content of the ultraviolet-curable resin in the material constituting the transparent resin member 30 is not particularly limited, but may be, for example, 100% or less. The expression that the material constituting the transparent resin member 30 contains 100% of an ultraviolet-curable resin means that the transparent resin member 30 is composed of the ultraviolet-curable resin except for unavoidable impurities or unintentional inclusions. The material constituting the transparent resin member 30 may be an ultraviolet-curable resin.

Examples of the ultraviolet-curable resin contained in the material constituting the transparent resin member 30 include, for example, epoxy-based resins and acrylic-based resins. Specifically, as the ultraviolet-curable resin, epoxy acrylate-based resins, urethane acrylate-based resins, or polyester acrylate-based resins can be used. Among these, preferably, the ultraviolet-curable resin contained in the material constituting the transparent resin member 30 is an epoxy-based resin. By using an epoxy-based resin as the ultraviolet-curable resin, the transparency of the transparent resin member 30 can be enhanced, making it easier for light to pass through the transparent resin member 30. As a result, the interior of a body cavity can be brightly illuminated by the light-emitting part 20, and it becomes possible to provide the endoscope 1 that facilitates observation of the interior of the body cavity.

As shown in FIG. 1, preferably, the endoscope 1 further includes a tubular body 40 having an inner lumen and capable of accommodating the imaging element 10, the transparent resin member 30, and the light-emitting part 20. By the endoscope 1 having the tubular body 40, its rigidity can be increased even if it is of a small diameter. Therefore, when the endoscope 1 is inserted into a body cavity to reach a target site, the imaging element 10, the transparent resin member 30, and the light-emitting part 20 can be housed in the inner lumen of the tubular body 40, thereby enhancing the pushability of the endoscope 1 and making insertion of the endoscope 1 into the body cavity easier.

The tubular body 40 is an elongated member having one end, the other end, and an inner lumen. Preferably, the imaging element 10, the transparent resin member 30, and the light-emitting part 20 can be accommodated in the inner lumen of the tubular body 40.

Examples of the cross-sectional shape of the outer contour of the tubular body 40 in a cross section perpendicular to the extending direction of the tubular body 40 include a circular shape, a polygonal shape, or a combination thereof. Preferably, the cross-sectional shape of the outer contour of the tubular body 40 in a cross section perpendicular to the extending direction of the tubular body 40 is circular. When the cross-sectional shape of the outer contour of the tubular body 40 is circular, the outer surface of the tubular body 40 becomes smooth, making it less likely to damage the wall of a biological lumen or the like even when the outer surface of the tubular body 40 comes into contact with it.

Preferably, the cross-sectional shape of the inner lumen of the tubular body 40 in a cross section perpendicular to the extending direction of the tubular body 40 is circular.

When the cross-sectional shape of the inner lumen of the tubular body 40 is circular, the surface of the inner lumen becomes smooth, making it easier for the tubular body 40 to slide smoothly over the imaging element 10, the transparent resin member 30, and the light-emitting part 20.

The number of inner lumens provided in the tubular body 40 may be one, but preferably, the tubular body 40 has a plurality of inner lumens. By the tubular body 40 having a plurality of inner lumens, it becomes possible to deliver and use various treatment tools in a body cavity while observing the interior of the body cavity with the endoscope 1. These treatment tools may be used to perform various treatments, such as injecting medication into a treatment target such as biological tissue in the body cavity; collecting, grasping, constricting, incising, excising, heating, ablating, performing phototherapy, ultrasonic therapy, or shockwave therapy on a treatment target.

Examples of materials constituting the tubular body 40 include synthetic resins such as polyolefin-based resins such as polyethylene and polypropylene; polyamide-based resins such as nylon; polyester-based resins such as PET; aromatic polyether ketone-based resins such as PEEK; polyether polyamide-based resins; polyurethane-based resins; polyimide-based resins; fluorine-based resins such as PTFE, PFA, and ETFE; and polyvinyl chloride-based resins. The tubular body 40 may have a single-layer structure or a multilayer structure. In the case of a multilayer structure, for example, a metal braid made of stainless steel, carbon steel, or a nickel-titanium alloy may be used as an intermediate layer of a resin tube constituting the tubular body 40. The material constituting the tubular body 40 may be a fluorine-based resin, or PTFE. When the material constituting the tubular body 40 is a fluorine-based resin, it is possible to improve the sliding property of the outer surface and impart appropriate rigidity, thereby enabling the endoscope 1 to be smoothly inserted into a body cavity.

The length of the tubular body 40 in the longitudinal axis direction can be selected to be appropriate for treatments using the endoscope 1. For example, the length of the tubular body 40 in the longitudinal axis direction may be from 500 mm or longer and 2000 mm or shorter. Note that the length of the tubular body 40 in the longitudinal axis direction refers to the length from the distal end 40d of the tubular body 40 to the proximal end of the tubular body 40.

As shown in FIG. 1, preferably, in a state in which the imaging element 10, the transparent resin member 30, and the light-emitting part 20 are exposed from the tubular body 40, the transparent resin member 30 has a contact surface on its outer surface, the contact surface being capable of contacting a wall of a biological lumen. The state in which the imaging element 10, the transparent resin member 30, and the light-emitting part 20 are exposed from the tubular body 40 refers to a condition in which they are located on a distal side relative to the distal end 40d of the tubular body 40 and are not disposed in the inner lumen of the tubular body 40. In the state in which the imaging element 10, the transparent resin member 30, and the light-emitting part 20 are exposed from the tubular body 40, the transparent resin member 30 having a contact surface on its outer surface, the contact surface being capable of contacting a wall of a biological lumen, results in the imaging element 10, the transparent resin member 30, and the light-emitting part 20 being in an uncovered state. Because the imaging element 10, the transparent resin member 30, and the light-emitting part 20 are in the uncovered state, when the endoscope 1 is placed in a biological lumen, no other element exists between the wall of the biological lumen and the imaging element 10, the transparent resin member 30, and the light-emitting part 20. As a result, light emitted from the light-emitting part 20 is less likely to be blocked by other elements, allowing the wall of the biological lumen to be brightly illuminated and thereby making it easier to observe the interior of the body cavity with the imaging element 10.

As shown in FIG. 1, a reflective material 50 that reflects light emitted from the light-emitting part 20 may be disposed inside the transparent resin member 30, on a proximal side relative to the imaging element 10 and on a distal side relative to the light-emitting part 20. That is, the reflective material 50 may be disposed inside the transparent resin member 30 and between the imaging element 10 and the light-emitting part 20 in the longitudinal axis direction of the endoscope 1. Disposing the reflective material 50 inside the transparent resin member 30, on a proximal side relative to the proximal end 10p of the imaging element 10 and on a distal side relative to the proximal end 20p of the light-emitting part 20, allows light emitted from the light-emitting part 20 toward the imaging element 10 to be reflected by the reflective material 50. Light emitted from the light-emitting part 20 toward the imaging element 10 tends to be blocked by the imaging element 10 and is less likely to contribute to the effect of illuminating a region distal to the distal end 1d of the endoscope 1. However, by being reflected by the reflective material 50, the light emitted from the light-emitting part 20 toward the imaging element 10 becomes more likely to be scattered in various directions as reflected light. As a result, light can be emitted around the distal end portion of the endoscope 1, making it possible to brightly illuminate the interior of the body cavity and thereby facilitating observation of the interior of the body cavity.

The surface of the reflective material 50 can be composed of, for example, aluminum, gold, silver, copper, tin, titanium dioxide, tantalum pentoxide, aluminum oxide, silicon dioxide, or magnesium fluoride. Among these, the surface of the reflective surface of the reflective material 50 may be composed of aluminum. By composing the surface of the reflective surface of the reflective material 50 of aluminum, the reflective material 50 becomes easier to handle, and it is possible to improve safety during the manufacturing or use of the endoscope 1.

The shape of the reflective material 50 is not particularly limited and can be, for example, a plate shape, a cylindrical shape, a prismatic shape, a circular tubular shape, a polygonal tubular shape, a truncated conical tubular shape, or a truncated pyramidal tubular shape. Among these, the shape of the reflective material 50 may be a truncated conical tubular shape, as shown in FIG. 1. By forming the shape of the reflective material 50 as a truncated conical tubular shape, the reflective material 50 becomes more likely to reflect light emitted from the light-emitting part 20, while the overall size of the reflective material 50 can be prevented from becoming too large, making it possible to reduce the diameter of the distal end portion of the endoscope 1.

When the shape of the reflective material 50 is a truncated conical tubular shape or a truncated pyramidal tubular shape, the reflective material 50 may be disposed, as shown in FIG. 1, outside a distal end portion of the light-emitting part 20, and the reflective surface of the reflective material 50 may be oriented to face a distal side. By disposing the reflective material 50 having a truncated conical tubular shape or a truncated pyramidal tubular shape outside the distal end portion of the light-emitting part 20 and by orienting the reflective surface of the reflective material 50 to face the distal side, the reflective material 50 becomes more likely to reflect light emitted from the light-emitting part 20, and the reflected light becomes more likely to be scattered in various directions.

An amount of ultraviolet light included in light emitted from the light-emitting part 20 may be 1 μW/cm² or less. By setting the amount of ultraviolet light included in light emitted from the light-emitting part 20 to 1 μW/cm² or less, when the light-emitting part 20 emits light during use of the endoscope 1 or the like, it is possible to reduce the amount of ultraviolet light irradiated onto the transparent resin member 30 that includes an ultraviolet-curable resin. As a result, the transparent resin member 30 becomes less likely to be cured again, and it is possible to obtain effects such as preventing the transparent resin member 30 from becoming excessively cured and brittle, and preventing the transparent resin member 30 from remaining in a deformed state, such as being bent.

The amount of ultraviolet light included in light emitted from the light-emitting part 20 may be 1.0 μW/cm² or less, 0.9 μW/cm² or less, or 0.8 μW/cm² or less. By setting the upper limit of the amount of ultraviolet light included in light emitted from the light-emitting part 20 within the above-described range, it becomes easier to reduce the amount of ultraviolet light irradiated onto the transparent resin member 30 when the light-emitting part 20 emits light. A lower limit of the amount of ultraviolet light included in light emitted from the light-emitting part 20 is not particularly limited, and may be, for example, 0 μW/cm² or more (including 0 μW/cm²). The amount of ultraviolet light included in light emitted from the light-emitting part 20 being 0 μW/cm² means that the light emitted from the light-emitting part 20 contains no ultraviolet light.

As shown in FIG. 1, a covering tube 60 may be provided on a proximal side relative to the distal end 20d of the light-emitting part 20. By providing the covering tube 60 on the proximal side relative to the distal end 20d of the light-emitting part 20, it becomes possible to enhance the sliding property of the outer surface of the endoscope 1 on the proximal side relative to the distal end 20d of the light-emitting part 20, and to make it easier to insert the endoscope 1.

Examples of materials constituting the covering tube 60 include polyamide-based resins, polyester-based resins, polyurethane-based resins, polyolefin-based resins, fluorine-based resins, vinyl chloride-based resins, silicone-based resins, natural rubber, and synthetic rubber. One of these materials may be used alone, or two or more of them may be used in combination. Among these, the material constituting the covering tube 60 may be a polyamide-based resin. By using a polyamide-based resin as the material constituting the covering tube 60, it is possible to enhance the slipperiness of the covering tube 60.

As shown in FIGS. 1 and 2, a core member 70 may be disposed inside the transparent resin member 30, and the core member 70 may extend from at least the proximal end 10p of the imaging element 10 to a position proximal to the distal end 60d of the covering tube 60. By disposing the core member 70 inside the transparent resin member 30 and by extending the core member 70 from at least the proximal end 10p of the imaging element 10 to a position proximal to the distal end 60d of the covering tube 60, it becomes possible to increase the rigidity of a distal end portion of the endoscope 1 with the core member 70, and to make it easier to insert the endoscope 1.

The core member 70 may be at least one of the wiring 110 of the imaging element 10 and the wiring 120 of the light-emitting part 20. When the core member 70 is at least one of the wiring 110 of the imaging element 10 and the wiring 120 of the light-emitting part 20, at least one of the wiring 110 of the imaging element 10 and the wiring 120 of the light-emitting part 20 also serves as the core member 70. By using the wiring 110 of the imaging element 10 or the wiring 120 of the light-emitting part 20 as the core member 70, it becomes possible to reduce the number and types of components that constitute the endoscope 1. As a result, it is possible to reduce the outer diameter of the endoscope 1 and to improve the low invasiveness of the endoscope 1. In order to use at least one of the wiring 110 of the imaging element 10 and the wiring 120 of the light-emitting part 20 as the core member 70, for example, the rigidity of the wiring 110 or 120 may be increased by increasing the thickness of a coating of at least one of the wiring 110 and the wiring 120, or by using a material with high hardness as the coating material.

Alternatively, the core member 70 may be a pull wire for bending a distal end portion of the endoscope 1. Specifically, a distal portion of the core member 70 may be fixed to at least one of the imaging element 10, the transparent resin member 30, and the light-emitting part 20, and a proximal portion of the core member 70 may extend to at least a proximal portion of the endoscope 1. By fixing the distal portion of the core member 70 to at least one of the imaging element 10, the transparent resin member 30, and the light-emitting part 20, and by extending the proximal portion of the core member 70 to at least the proximal portion of the endoscope 1, it becomes possible to bend the distal end portion of the endoscope 1 by pulling the core member 70 toward the proximal side. As a result, it becomes easier to control the position of the distal end portion of the endoscope 1 in a body cavity, thereby facilitating observation inside the body cavity.

The number of the core member 70 provided in the endoscope 1 may be one, or may be two or more. When the number of the core member 70 provided in the endoscope 1 is one, it is possible to reduce the number of components constituting the endoscope 1 and to achieve a smaller outer diameter of the endoscope 1. When the number of the core member 70 provided in the endoscope 1 is two or more, for example, by pulling one of the core members 70 toward the proximal side, the distal end portion of the endoscope 1 can be bent in one direction, and by pulling another core member 70 different from the one, the distal end portion of the endoscope 1 can be bent in a direction different from the one direction. As a result, it becomes easier to control the position of the distal end portion of the endoscope 1.

As shown in FIGS. 3 and 4, at least one of the imaging element 10 and the light-emitting part 20 may have a groove 80 formed on its surface. By forming the groove 80 on the surface of at least one of the imaging element 10 and the light-emitting part 20, the contact area between the imaging element 10 or the light-emitting part 20 and the transparent resin member 30 increases. As a result, the bonding strength between the imaging element 10 or the light-emitting part 20 and the transparent resin member 30 can be enhanced, and the imaging element 10 and the light-emitting part 20 can be less likely to come off from the transparent resin member 30.

Examples of the groove 80 formed on a surface of at least one of the imaging element 10 and the light-emitting part 20 include a groove that extends in a direction along the longitudinal axis direction of the endoscope 1, a groove that extends in a direction perpendicular to the longitudinal axis direction of the endoscope 1, and a groove that extends in a direction oblique to the longitudinal axis direction of the endoscope 1. The groove 80 may extend in a straight line, or may extend in a curved line. Furthermore, the groove 80 may be formed continuously or intermittently on a surface of at least one of the imaging element 10 and the light-emitting part 20.

Among these, at least one of the imaging element 10 and the light-emitting part 20 may have the groove 80 formed on its surface, the groove 80 extending in a direction perpendicular to the longitudinal axis direction of the endoscope 1. By forming the groove 80 on the surface of at least one of the imaging element 10 and the light-emitting part 20 so as to extend in a direction perpendicular to the longitudinal axis direction of the endoscope 1, it becomes possible to strengthen the bonding between the imaging element 10 or the light-emitting part 20 and the transparent resin member 30 even when the distal end portion of the endoscope 1 is bent, and to make the imaging element 10 and the light-emitting part 20 less likely to come off from the transparent resin member 30.

The number of the groove 80 formed on at least one of the imaging element 10 and the light-emitting part 20 may be plural. When the number of the groove 80 is plural, the area of the surface of the imaging element 10 or the light-emitting part 20 in contact with the transparent resin member 30 can be increased, and the bonding strength between the imaging element 10 or the light-emitting part 20 and the transparent resin member 30 can be more easily enhanced. When the number of the groove 80 formed on at least one of the imaging element 10 and the light-emitting part 20 is plural, the width, depth, length, and extending direction of each of the groove 80 may be the same or may be different.

The groove 80 may be formed on each of the surfaces of the imaging element 10 and the light-emitting part 20. In other words, the groove 80 may be formed on the surfaces of both the imaging element 10 and the light-emitting part 20. By forming the groove 80 on each of the surfaces of the imaging element 10 and the light-emitting part 20, the area of contact between the imaging element 10 and the transparent resin member 30, and between the light-emitting part 20 and the transparent resin member 30 can be increased, thereby making both the imaging element 10 and the light-emitting part 20 less likely to come off from the transparent resin member 30.

As shown in FIGS. 2 to 4, in a cross section perpendicular to a longitudinal axis direction of the endoscope 1, the imaging element 10 and the light-emitting part 20 each have a rectangular cross-sectional shape, and the transparent resin member 30 has a circular cross-sectional shape. By the imaging element 10 and the light-emitting part 20 each having a rectangular cross-sectional shape, the area of contact of the surface of the imaging element 10 and the surface of the light-emitting part 20 with the transparent resin member 30 can be increased. As a result, the bonding strength between the imaging element 10 and the transparent resin member 30, and between the light-emitting part 20 and the transparent resin member 30 can be enhanced, making the imaging element 10 and the light-emitting part 20 less likely to come off from the transparent resin member 30. In addition, by the transparent resin member 30 having a circular cross-sectional shape, the outer surface of the transparent resin member 30 becomes smooth, and when the transparent resin member 30 comes into contact with other objects such as the wall of a biological lumen, it is less likely to damage such objects, thereby enabling the endoscope 1 to provide improved safety.

In confirming the cross-sectional shape of the imaging element 10 in a cross section perpendicular to a longitudinal axis direction of the endoscope 1, when the groove 80 is formed on a surface of the imaging element 10, the confirmation of the cross-sectional shape shall be made assuming that the groove 80 does not exist. That is, for example, when the groove 80 extending in a direction perpendicular to the longitudinal axis direction of the endoscope 1 is formed on the surface of the imaging element 10, as shown by the dashed line in FIG. 3, the cross-sectional shape shall be confirmed assuming that the indentation of the groove 80 is filled. The same applies to the light-emitting part 20. In confirming the cross-sectional shape of the light-emitting part 20 in a cross section perpendicular to the longitudinal axis direction of the endoscope 1, when the groove 80 is formed on a surface of the light-emitting part 20, the confirmation of the cross-sectional shape shall be made assuming that the groove 80 does not exist.

An average value of a surface roughness Ra of the transparent resin member 30 may be 25 μm or less. The surface roughness Ra of the transparent resin member 30 refers to an arithmetic average roughness Ra measured along a roughness profile in the longitudinal axis direction on an outer peripheral surface of the transparent resin member 30 over a reference length. The arithmetic average roughness Ra corresponds to the arithmetic average roughness Ra defined in JIS B 0601 (2001), and is measured in accordance with JIS B 0633 (2001). A measuring instrument defined in JIS B 0651 (2001) is used for the measurement of the arithmetic average roughness Ra. The average value of the surface roughness Ra of the transparent resin member 30 is defined as an average of the Ra values measured at ten or more measurement points set along the longitudinal axis direction of the transparent resin member 30.

When the average value of the surface roughness Ra of the transparent resin member 30 is 25 μm or less, the surface of the transparent resin member 30 becomes smooth. As a result, even when the transparent resin member 30 comes into contact with another object such as a wall of a biological lumen, it is less likely to damage the object, thereby improving the safety of the endoscope 1.

The average value of the surface roughness Ra of the transparent resin member 30 may be 25 μm or less, 23 μm or less, or 20 μm or less. By setting the upper limit of the average value of the surface roughness Ra of the transparent resin member 30 within the above range, the surface of the transparent resin member 30 can be made smooth. Although the lower limit of the average value of the surface roughness Ra of the transparent resin member 30 is not particularly limited, it may be, for example, 0 μm or more, 0.5 μm or more, or 1 μm or more.

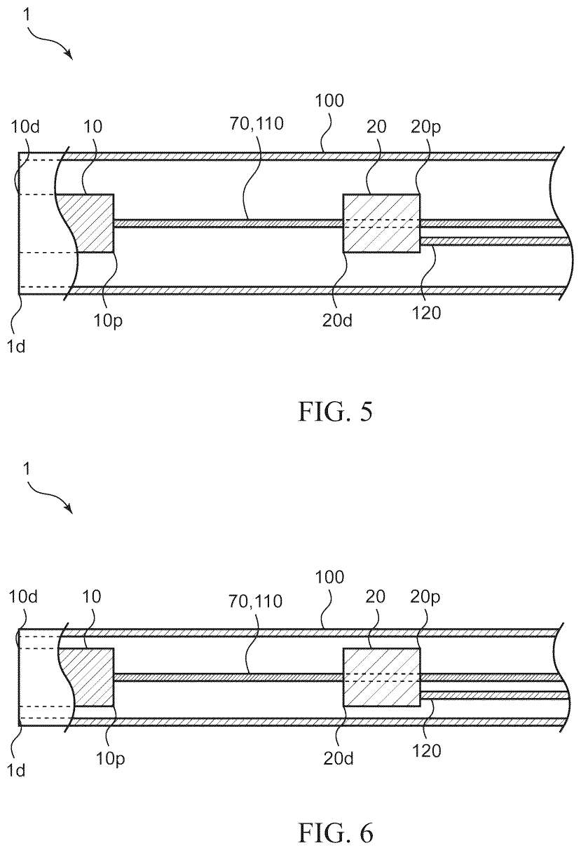

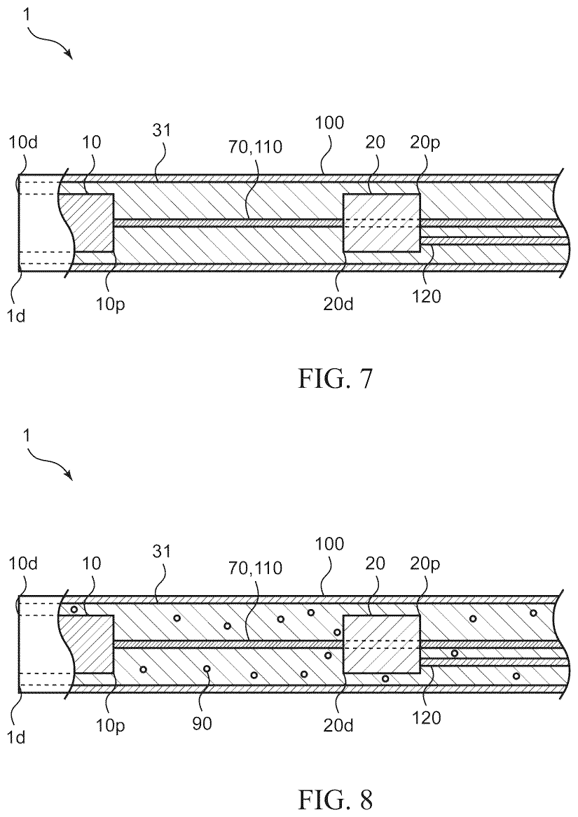

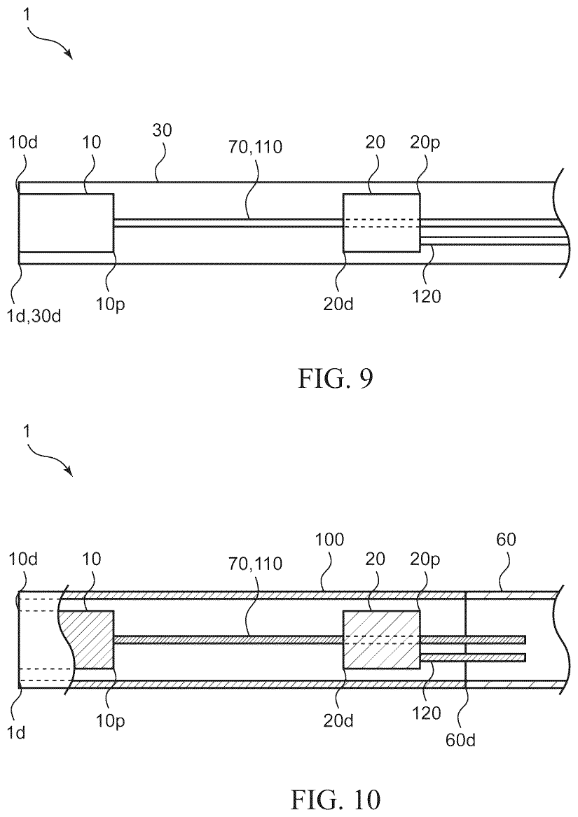

Next, a method for manufacturing an endoscope of one or more embodiments of the present invention will be described. In the following description of the manufacturing method of the endoscope, overlapping portions with the above description of the endoscope will be omitted. FIG. 5 is a schematic view (partially in sectional view) illustrating a step of placing the imaging element 10 and the light-emitting part 20 in an inner lumen of a heat-shrinkable tube 100 in one or more embodiments of the present invention. FIG. 6 is a schematic view (partially in sectional view) illustrating a step of heating the heat-shrinkable tube 100. FIG. 7 is a schematic view (partially in sectional view) illustrating a step of filling a transparent liquid resin 31 into the inner lumen of the heat-shrinkable tube 100. FIG. 8 is a schematic view (partially in sectional view) illustrating a step of forming bubbles 90 in the transparent liquid resin 31. FIG. 9 is a schematic view illustrating a step of removing the heat-shrinkable tube 100. FIG. 10 is a schematic view (partially in sectional view) illustrating a step of placing a wire 110 of the imaging element 10 and a wire 120 of the light-emitting part 20 in an inner lumen of a covering tube 60.

As shown in FIG. 5, the method for manufacturing an endoscope 1 includes a step of placing the imaging element 10 and the light-emitting part 20 in an inner lumen of a heat-shrinkable tube 100. This step may hereinafter be referred to as a placement step. The imaging element 10 is placed in the inner lumen of the heat-shrinkable tube 100 such that the imaging element 10 is located on a distal side relative to the light-emitting part 20.

In the placement step, the reflective material 50 may be placed in the inner lumen of the heat-shrinkable tube 100 between the imaging element 10 and the light-emitting part 20.

As shown in FIG. 6, the method for manufacturing the endoscope 1 includes a step of heating the heat-shrinkable tube 100. This step may hereinafter be referred to as a heating step. By heating the heat-shrinkable tube 100, the heat-shrinkable tube 100 shrinks in diameter.

The heating step may be performed after the placement step. That is, after placing the imaging element 10 and the light-emitting part 20 in the inner lumen of the heat-shrinkable tube 100, the heat-shrinkable tube 100 is heated. By performing the heating step after the placement step, it becomes easier to temporarily fix the positions of the imaging element 10 and the light-emitting part 20 using the heat-shrinkable tube 100 that has shrunk in diameter.

As shown in FIG. 7, the method for manufacturing the endoscope 1 includes a step of filling the inner lumen of the heat-shrinkable tube 100 with a transparent liquid resin 31 that is curable by ultraviolet irradiation. This step may hereinafter be referred to as a filling step.

In the filling step, the imaging element 10 and the light-emitting part 20, which are disposed in the inner lumen of the heat-shrinkable tube 100, come into contact with the transparent liquid resin 31.

The filling step may be performed after the heating step. By performing the filling step after the heating step, it is possible to prevent the transparent liquid resin 31 from flowing into unintended areas, thereby improving the manufacturing efficiency of the endoscope 1.

The method may include a step of moving the heat-shrinkable tube 100 toward the side where the imaging element 10 is disposed, before the filling step. Preferably, even after the heat-shrinkable tube 100 has been moved toward the imaging element 10, it remains in a state in which the imaging element 10 and the light-emitting part 20 are disposed in the inner lumen of the heat-shrinkable tube 100. By including the step of moving the heat-shrinkable tube 100 toward the imaging element 10 before the filling step, it becomes easier to pour the transparent liquid resin 31 into the inner lumen of the heat-shrinkable tube 100 from the side where the light-emitting part 20 is disposed in the filling step, thereby making it less likely for the transparent liquid resin 31 to flow beyond the imaging element 10 and cause a manufacturing defect in the endoscope 1, or for an excessive amount of the transparent liquid resin 31 to be poured. As a result, the endoscope 1 can be manufactured more efficiently. Furthermore, in a case where the endoscope 1 has the covering tube 60 on a proximal side relative to the distal end 20d of the light-emitting part 20, the step of moving the heat-shrinkable tube 100 toward the imaging element 10 before the filling step also makes it easier for the transparent liquid resin 31 to be filled into the inner lumen of the covering tube 60.

As shown in FIG. 8, the method for manufacturing the endoscope 1 includes a step of forming the bubbles 90 in the transparent liquid resin 31. This step may hereinafter be referred to as a bubble-forming step. In FIG. 8, the bubbles 90 are illustrated in an enlarged manner for explanation of the bubble-forming step.

The bubble-forming step may be performed after the filling step or before the filling step. Specifically, after filling the transparent liquid resin 31 into the inner lumen of the heat-shrinkable tube 100, the bubbles 90 may be formed in the transparent liquid resin 31 within the inner lumen of the heat-shrinkable tube 100. Alternatively, after forming the bubbles 90 in the transparent liquid resin 31, the transparent liquid resin 31 containing the bubbles 90 may be filled into the inner lumen of the heat-shrinkable tube 100.

Among these, the bubble-forming step may be performed after the filling step. By performing the bubble-forming step after the filling step, it becomes easier for the transparent liquid resin 31 to be poured into every corner of the inner lumen of the heat-shrinkable tube 100, thereby facilitating sufficient filling within the heat-shrinkable tube 100. As a result, the transparent liquid resin 31 can more easily spread throughout the inner lumen of the heat-shrinkable tube 100, making it less likely for molding defects of the transparent resin member 30 to occur.

Examples of methods for forming the bubbles 90 in the transparent liquid resin 31 include: stirring the transparent liquid resin 31 with a rod-shaped instrument having blade-like parts such as a propeller or with a whisk-like instrument to incorporate air or other gases into the transparent liquid resin 31; injecting gas into the transparent liquid resin 31 by inserting a syringe, air pump, or the like; or adding a foaming agent to the transparent liquid resin 31 to generate foam. Among these, a preferable method for forming the bubbles 90 in the transparent liquid resin 31 is to stir the transparent liquid resin 31 with a rod-shaped instrument or the like to incorporate air. By forming the bubbles 90 in the transparent liquid resin 31 through air incorporation by stirring, it becomes easier to form the bubbles 90 having a major axis of 50 μm or more and 500 μm or less in the region A1.

The method for manufacturing the endoscope 1 includes a step of forming the transparent resin member 30 by irradiating the transparent liquid resin 31 with ultraviolet light. This step may hereinafter be referred to as a forming step. The forming step is performed after the filling step. That is, by irradiating the transparent liquid resin 31 with ultraviolet light after the transparent liquid resin 31 is filled into the inner lumen of the heat-shrinkable tube 100, the transparent liquid resin 31 is cured, and the transparent resin member 30 can be formed.

As shown in FIG. 9, the method for manufacturing the endoscope 1 includes a step of removing the heat-shrinkable tube 100. This step may hereinafter be referred to as a removal step. The removal step is performed after the forming step. In the forming step, the transparent liquid resin 31 filled in the inner lumen of the heat-shrinkable tube 100 is cured by ultraviolet irradiation to form the transparent resin member 30. That is, the heat-shrinkable tube 100 is present outside the transparent resin member 30 formed in the forming step. In the endoscope 1, if the heat-shrinkable tube 100 remains on the outside of the transparent resin member 30, there is a risk that light emitted from the light-emitting part 20 may have difficulty passing through the transparent resin member 30, or that the sliding property of the outer surface of the transparent resin member 30 may deteriorate, making it more difficult for it to pass through a biological lumen. Therefore, in the removal step, the heat-shrinkable tube 100 present outside the transparent resin member 30 is removed.

In one or more embodiments of the present invention, the transparent resin member 30 has one or more and ten or fewer of the bubbles 90, each having a major axis of 50 μm or more and 500 μm or less, in the region A1 having a length of 0.1 mm in a longitudinal axis direction of the endoscope 1; however, the bubbles 90 are omitted from illustration in FIG. 9.

As a method for removing the heat-shrinkable tube 100 in the removal step, examples include: making an incision in the heat-shrinkable tube 100 using a knife or the like and removing the heat-shrinkable tube 100; scraping off the heat-shrinkable tube 100 using a file or the like; and bringing the heat-shrinkable tube 100 into contact with a fluid that dissolves the heat-shrinkable tube 100 for removal.

By including the placement step, the heating step, the filling step, the bubble forming step, the forming step, and the removal step in the manufacturing method for the endoscope 1, it becomes possible to manufacture the endoscope 1 in which the transparent resin member 30 has one or more and ten or fewer of the bubbles 90, each having a major axis of 50 μm or more and 500 μm or less, in the region A1 having a length of 0.1 mm in a longitudinal axis direction of the endoscope 1. When the transparent resin member 30 has one or more and ten or fewer of the bubbles 90 in the region A1, light emitted from the light-emitting part 20 is diffused by the bubbles 90 present in the transparent resin member 30 as the light passes through the transparent resin member 30. As a result, the light emitted from the light-emitting part 20 can easily illuminate a wide area within a body cavity, which makes it possible to manufacture the endoscope 1 that facilitates observation of the interior of the body cavity.

Preferably, the heat-shrinkable tube 100 is capable of transmitting ultraviolet light, and in the forming step of irradiating the transparent liquid resin 31 with ultraviolet light to form the transparent resin member 30, the ultraviolet light is irradiated from outside the heat-shrinkable tube 100. By irradiating the ultraviolet light from outside the heat-shrinkable tube 100 in the forming step, it becomes easier to irradiate the transparent liquid resin 31 filled in the inner lumen of the heat-shrinkable tube 100 with ultraviolet light. As a result, formation of the transparent resin member 30 can be facilitated, and the efficiency of the forming step can be improved.

The heat-shrinkable tube 100 may be capable of transmitting 50% or more of the irradiated ultraviolet light, 60% or more, or 70% or more. By setting the lower limit of the transmission ratio of ultraviolet light through the heat-shrinkable tube 100 within the above range, it becomes possible to shorten the time required to cure the transparent liquid resin 31 by irradiating the transparent liquid resin 31 with ultraviolet light to form the transparent resin member 30, and to improve the efficiency of the forming step. The upper limit of the transmission ratio of ultraviolet light through the heat-shrinkable tube 100 is not particularly limited, and may be, for example, 100% or less.

Preferably, in the step of filling the inner lumen of the heat-shrinkable tube 100 with the transparent liquid resin 31, the core member 70 is disposed in the inner lumen of the heat-shrinkable tube 100, and in the step of forming the bubbles 90 in the transparent liquid resin 31, the core member 70 is moved. By disposing the core member 70 in the inner lumen of the heat-shrinkable tube 100 in the filling step and moving the core member 70, which is disposed in the inner lumen of the heat-shrinkable tube 100, in the bubble forming step, the bubbles 90 can be formed in the transparent liquid resin 31 in the inner lumen of the heat-shrinkable tube 100 by the movement of the core member 70. Accordingly, the bubble forming step can be efficiently performed, and the manufacturing efficiency of the endoscope 1 can be improved.

The movement of the core member 70 in the bubble forming step may be movement in a longitudinal direction of the heat-shrinkable tube 100, movement in a radial direction of the heat-shrinkable tube 100, or movement in a circumferential direction of the heat-shrinkable tube 100. The direction of movement of the core member 70 in the bubble forming step may be any one of these directions or a direction that combines two or more of them. Among these, the movement of the core member 70 in the bubble forming step may be in a direction including at least one of a radial direction component and a circumferential direction component of the heat-shrinkable tube 100. That is, in the bubble forming step, it is preferable to move the core member 70 in at least one of the radial direction and the circumferential direction of the heat-shrinkable tube 100. When the movement of the core member 70 in the bubble forming step is in a direction including at least one of the radial direction component and the circumferential direction component of the heat-shrinkable tube 100, it becomes possible to form the bubbles 90 in the transparent liquid resin 31 in a state in which a load is less likely to be applied to the core member 70.

As shown in FIG. 10, the method for manufacturing the endoscope 1 may include: before filling the inner lumen of the heat-shrinkable tube 100 with the transparent liquid resin 31, a step of placing the covering tube 60 on a proximal side relative to the light-emitting part 20 and a step of inserting the wire 110 of the imaging element 10 and the wire 120 of the light-emitting part 20 into an inner lumen of the covering tube 60; and a step of filling the transparent liquid resin 31 into the inner lumen at the distal end 60d of the covering tube 60 before irradiating the transparent liquid resin 31 with ultraviolet light to form the transparent resin member 30. Hereinafter, the step of placing the covering tube 60 on the proximal side relative to the light-emitting part 20 may be referred to as the covering tube placement step; the step of inserting the wire 110 of the imaging element 10 and the wire 120 of the light-emitting part 20 into the inner lumen of the covering tube 60 may be referred to as the wire insertion step; and the step of filling the transparent liquid resin 31 into the inner lumen at the distal end 60d of the covering tube 60 may be referred to as the covering tube filling step.

That is, in the method for manufacturing the endoscope 1, it is preferable to perform the filling step of filling the inner lumen of the heat-shrinkable tube 100 with the transparent liquid resin 31 after performing the covering tube placement step and the wire insertion step, and to perform the forming step after performing the covering tube filling step of filling the transparent liquid resin 31 into the inner lumen at the distal end 60d of the covering tube 60. By performing filling step after performing the covering tube placement step and the wire insertion step, it becomes easier to align the positions of the wire 110 of the imaging element 10 and the wire 120 of the light-emitting part 20 and to fill the inner lumen of the heat-shrinkable tube 100 with the transparent liquid resin 31. Furthermore, by performing forming step after performing the covering tube filling step of filling the transparent liquid resin 31 into the inner lumen at the distal end 60d of the covering tube 60, the transparent resin member 30 can extend to the inner lumen at the distal end 60d of the covering tube 60, which enhances the bonding strength between the transparent resin member 30 and the covering tube 60 and makes the transparent resin member 30 less likely to detach from the covering tube 60.

It is preferable to perform both the covering tube placement step and the wire insertion step before the filling step. The covering tube placement step may be performed before the wire insertion step or after the wire insertion step. That is, the step of placing the covering tube 60 on a proximal side relative to the light-emitting part 20 may be performed after the step of inserting the wire 110 of the imaging element 10 and the wire 120 of the light-emitting part 20 into the inner lumen of the covering tube 60, or the step of inserting the wire 110 of the imaging element 10 and the wire 120 of the light-emitting part 20 into the inner lumen of the covering tube 60 may be performed after the step of placing the covering tube 60 on a proximal side relative to the light-emitting part 20.

The covering tube filling step may be performed either before or after the filling step. That is, the step of filling the inner lumen of the covering tube 60 at the distal end 60d with the transparent liquid resin 31 may be performed after the step of filling the inner lumen of the heat-shrinkable tube 100 with the transparent liquid resin 31, or the step of filling the inner lumen of the heat-shrinkable tube 100 with the transparent liquid resin 31 may be performed after the step of filling the inner lumen of the covering tube 60 at the distal end 60d with the transparent liquid resin 31.

The covering tube filling step may be performed after the covering tube placement step and the wire insertion step. That is, it is preferable to perform the step of filling the inner lumen at the distal end 60d of the covering tube 60 with the transparent liquid resin 31 after performing the step of placing the covering tube 60 on the proximal side relative to the light-emitting part 20 and the step of inserting the wire 110 of the imaging element 10 and the wire 120 of the light-emitting part 20 into the inner lumen of the covering tube 60. By performing the covering tube filling step after the covering tube placement step and the wire insertion step, the transparent liquid resin 31 is filled into the inner lumen at the distal end 60d of the covering tube 60 while the wire 110 of the imaging element 10 and the wire 120 of the light-emitting part 20 are inserted in the inner lumen of the covering tube 60, and thus the transparent liquid resin 31 can more easily stay in the inner lumen at the distal end 60d of the covering tube 60.

It is preferable to perform the step of filling the inner lumen of the heat-shrinkable tube 100 with the transparent liquid resin 31 and the step of irradiating the transparent liquid resin 31 with ultraviolet light to form the transparent resin member 30 under a reduced pressure condition. By performing the filling step and the forming step under a reduced pressure condition, it becomes easier to adjust the number and size of the bubbles 90 present in the transparent liquid resin 31, thereby making it easier to obtain the transparent resin member 30 having a desired structure.

It is also preferable to perform the step of filling the transparent liquid resin 31 into the inner lumen at the distal end 60d of the covering tube 60 under a reduced pressure condition. By performing the covering tube filling step under a reduced pressure condition, it becomes easier to adjust the bubbles 90 in the transparent liquid resin 31 filled in the inner lumen at the distal end 60d of the covering tube 60.

The present application claims priority based on Japanese Patent Application No. 2023-000667 filed on Jan. 5, 2023. All the contents described in Japanese Patent Application No. 2023-000667 filed on Jan. 5, 2023 are incorporated herein by reference.

### DESCRIPTION OF REFERENCE SIGNS

1: endoscope
1d: distal end of endoscope
10: imaging element
10d: distal end of imaging element
10p: proximal end of imaging element
20: light-emitting part
20d: distal end of light-emitting part
20p: proximal end of light-emitting part
30: transparent resin member
30d: distal end of transparent resin member
30p: proximal end of transparent resin member
30A: central region
30B: peripheral region
30C: distal region
23D: proximal region
30E: intermediate region
31: transparent liquid resin
40: tubular body
40d: distal end of tubular body
50: reflective material
60: covering tube
60d: distal end of covering tube
70: core member
80: groove
90: bubbles
100: heat-shrinkable tube
110: wire of imaging element
120: wire of light-emitting part
A1: region having a length of 0.1 mm in longitudinal axis direction of endoscope
P1: centroid of outer shape of transparent resin member

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

1. An endoscope comprising:
an imaging element;
a light-emitting part; and
a transparent resin member that is fixed to the imaging element and the light-emitting part and through which light emitted from the light-emitting part is capable of passing,
wherein the transparent resin member has, in a region having a length of 0.1 mm in a longitudinal axis direction of the endoscope, one or more and ten or fewer bubbles each having a major axis of 50 μm or more and 500 μm or less.

2. The endoscope according to claim 1, wherein:
in a cross section perpendicular to the longitudinal axis direction at a midpoint of a length of the transparent resin member in the longitudinal axis direction of the endoscope, the transparent resin member comprises:
a central region defined by a circle centered at a centroid of an outer shape of the transparent resin member and having a diameter equal to half of a minor axis length of the outer shape of the transparent resin member, and
a peripheral region excluding the central region; and
a number of the bubbles present in the central region is greater than a number of the bubbles present in the peripheral region.

3. The endoscope according to claim 1, wherein:
the transparent resin member is located on a proximal side relative to a distal end of the imaging element and on a distal side relative to a proximal end of the light-emitting part;
the transparent resin member comprises:
a distal region positioned on a side of the imaging element,
a proximal region positioned on a side of the light-emitting part, and
an intermediate region positioned proximal to the distal region and distal to the proximal region;
a number of the bubbles present in the intermediate region is greater than a number of the bubbles present in the distal region; and
a number of the bubbles present in the proximal region is greater than a number of the bubbles present in the intermediate region.

4. The endoscope according to claim 1, wherein a material constituting the transparent resin member comprises an ultraviolet-curable resin.

5. The endoscope according to claim 1, further comprising a tubular body having an inner lumen and capable of accommodating the imaging element, the transparent resin member, and the light-emitting part in the inner lumen,
wherein, in a state in which the imaging element, the transparent resin member, and the light-emitting part are exposed from the tubular body, the transparent resin member has a contact surface on its outer surface, the contact surface being capable of contacting a wall of a biological lumen.

6. The endoscope according to claim 1, wherein a reflective material that reflects light emitted from the light-emitting part is disposed inside the transparent resin member, the reflective material being located on a proximal side relative to the imaging element and on a distal side relative to the light-emitting part.

7. The endoscope according to claim 1, wherein an amount of ultraviolet light comprised in light emitted from the light-emitting part is 1 μW/cm² or less.

8. The endoscope according to claim 1, further comprising:
a covering tube located on a proximal side relative to a distal end of the light-emitting part, and
a core member disposed inside the transparent resin member,
wherein the core member extends from at least a proximal end of the imaging element to a position proximal to a distal end of the covering tube.

9. The endoscope according to claim 1, wherein at least one of the imaging element and the light-emitting part has a groove that extends in a direction perpendicular to the longitudinal axis direction of the endoscope.

10. The endoscope according to claim 1, wherein, in a cross section perpendicular to the longitudinal axis direction of the endoscope, the imaging element and the light-emitting part each have a rectangular cross-sectional shape, and the transparent resin member has a circular cross-sectional shape.

11. The endoscope according to claim 1, wherein an average value of a surface roughness Ra of the transparent resin member is 25 μm or less.

12. A method for manufacturing an endoscope,
the endoscope comprising:
an imaging element,
a light-emitting part, and
a transparent resin member through which light emitted from the light-emitting part is capable of passing and which has, in a region having a length of 0.1 mm in a longitudinal axis direction, one or more and ten or fewer bubbles each having a major axis of 50 μm or more and 500 μm or less,
the method comprising:
placing the imaging element and the light-emitting part in an inner lumen of a heat-shrinkable tube;
heating the heat-shrinkable tube;
filling the inner lumen of the heat-shrinkable tube with a transparent liquid resin that is curable by ultraviolet irradiation;
forming bubbles in the transparent liquid resin;
irradiating the transparent liquid resin with ultraviolet light to form the transparent resin member; and
removing the heat-shrinkable tube.

13. The manufacturing method according to claim 12, wherein:
the heat-shrinkable tube is capable of transmitting ultraviolet light, and
in the step of irradiating the transparent liquid resin with ultraviolet light to form the transparent resin member, the ultraviolet light is irradiated from outside the heat-shrinkable tube.

14. The manufacturing method according to claim 12, wherein,
in the step of filling the inner lumen of the heat-shrinkable tube with the transparent liquid resin, a core member is disposed in the inner lumen of the heat-shrinkable tube, and
in the step of forming bubbles in the transparent liquid resin, the core member is moved.

15. The manufacturing method according to claim 12, further comprising:
before filling the inner lumen of the heat-shrinkable tube with the transparent liquid resin, placing a covering tube on a proximal side relative to the light-emitting part and inserting a wire of the imaging element and a wire of the light-emitting part into an inner lumen of the covering tube, and
before irradiating the transparent liquid resin with ultraviolet light to form the transparent resin member, filling the transparent liquid resin into the inner lumen at a distal end of the covering tube.
